(19) United States
(12) Patent Application Publication
(10) Pub. No.: US 2006/0184225 A1
(43) Pub. Date: Aug. 17, 2006

(54) Force distributing system for delivering a self-expanding stent

(75) Inventor: Jack Pryor, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/056,816

(22) Filed: Feb. 11, 2005

(51) Int. Cl. A61F 2/06

(52) U.S. Cl. 623/1.11

(57) ABSTRACT

The invention provides a system that distributes along the length of a stent those forces exerted on the stent during release of the stent from a sheath. The system includes a catheter inner member, a stent, and a sheath. Multiple longitudinally spaced protrusions extend from the outer surface of a distal portion of the inner member. Complementary longitudinally spaced apertures are formed in the wall of the stent. The stent is mounted on the inner member with the inner member protrusions received within the stent apertures. The sheath encloses the stent and is movable with respect to the stent. The system is assembled by aligning the protrusions and apertures and radially compressing the stent about the inner member. The resulting interlocked protrusions and apertures allow the stent to be withdrawn from a radial compression device into the sheath by pulling on the inner member.

### TECHNICAL FIELD

This invention relates generally to biomedical systems for treating vascular conditions and to methods for manufacturing and using such biomedical systems. More specifically, the invention relates to a stent delivery system that distributes along the length of a stent, forces exerted on the stent during release of the stent from a sheath and to methods for assembling and using such a system.

### BACKGROUND OF THE INVENTION

Stents are cylindrical devices that are radially expandable to hold open a segment of a vessel or other anatomical lumen after deployment in the lumen. Various types of stents are in use, including balloon expandable and self-expanding stents. Balloon expandable stents generally are conveyed to the area to be treated on balloon catheters. A self-expanding stent is conveyed to a treatment site while compressed within a sheath. Once positioned, the sheath is retracted, allowing expansion of the stent.

Before deployment of the self-expanding stent, the sheath exerts a uniform compressive force on the stent that retains the stent in an unexpanded or crimped (compressed) configuration. During deployment of the stent, an axial force caused by the withdrawal of the sheath adds to the compressive force already present in the sheath material. Typically, when the sheath is retracted to deploy the self-expanding stent, a stent stop on the inner member prevents the proximal end of the stent (the end nearest to the treating clinician) from moving past the stop, and the axial retraction forces are concentrated at the proximal end of the stent. This can result in crumpling or buckling of the stent (sometimes referred to as a “train wreck”), reducing the effective length of the stent or even causing it to fail.

Therefore, it would be desirable to have an improved system to deploy a self-expanding stent in a body lumen and methods for assembling and using such a treatment system that overcome the aforementioned and other disadvantages.

### SUMMARY OF THE INVENTION

One aspect according to the present invention is a system for treating a vascular condition. The system comprises a catheter inner member, a stent, and a sheath. The catheter inner member has a proximal portion and a distal portion, with the distal portion having a plurality of longitudinally spaced protrusions extending from an outer surface of the distal portion. The stent has a plurality of longitudinally spaced apertures formed in the wall of the stent. The stent is mounted on the inner member such that the inner member protrusions are received within the stent apertures. The sheath encloses the stent and is movable with respect to the stent.

Another aspect according to the present invention is a system for treating a vascular condition comprising a catheter, a stent disposed on the catheter, and a sheath releasably enclosing the stent. The system further comprises means for distributing along the length of the stent, forces that are exerted on the stent during release of the stent from the sheath.

Yet another aspect according to the present invention is a method of assembling a system for treating a vascular condition. A catheter inner member distal portion is positioned within a stent. The inner member distal portion has a plurality of longitudinally spaced protrusions, and the stent has a plurality of longitudinally spaced apertures. The inner member protrusions are configured to be aligned with the stent apertures. The stent is radially compressed about the inner member distal portion such that each inner member protrusion is received within a stent aperture. The stent and some or all of the inner member distal portion are positioned within a sheath.

Still another aspect according to the present invention is a method of treating a vascular condition. A sheathed stent is delivered to a target region of a vessel via a catheter. The sheath is retracted from the stent. Sheath retraction forces exerted on the stent during retraction of the sheath are distributed along the length of the stent.

The aforementioned and other features and advantages of the invention will become further apparent from the following detailed description, read in conjunction with the accompanying drawings, which are not to scale. The detailed description and drawings are merely illustrative of embodiments according to the invention rather than limiting.

### BRIEF DESCRIPTION OF THE DRAWINGS

FIG. 1 is an illustration of one embodiment of a system for treating a vascular condition, in accordance with the present invention;

FIG. 2 is an enlarged view of a protrusion extending from a distal portion of the inner member of the system of FIG. 1;

FIG. 2A is a cross sectional view of an end of an inner member through a location where protrusions from the inner member are 180 degrees apart;

FIG. 3 is a plan view of the stent of the system of FIG. 1, showing the stent cut longitudinally and laid flat;

FIG. 4 is a plan view of an alternative stent, in accordance with the present invention;

FIG. 5 is an enlarged view of an aperture formed in the wall of the stent of FIGS. 1 and 3, the aperture being formed between crowns of the stent;

FIG. 6 is an enlarged view of an aperture formed in the wall of an alternative stent, the aperture being formed between crowns of the stent;

FIG. 7 is an enlarged view of an aperture formed in the wall of an alternative stent, the aperture being formed within a crown of the stent;

FIG. 8 is a flow diagram of one embodiment of a method of assembling a system for treating a vascular condition, in accordance with the present invention; and

FIG. 9 is a flow diagram of one embodiment of a method of treating a vascular condition, in accordance with the present invention.

Like reference numbers are used throughout the drawings to refer to like parts.

### DETAILED DESCRIPTION

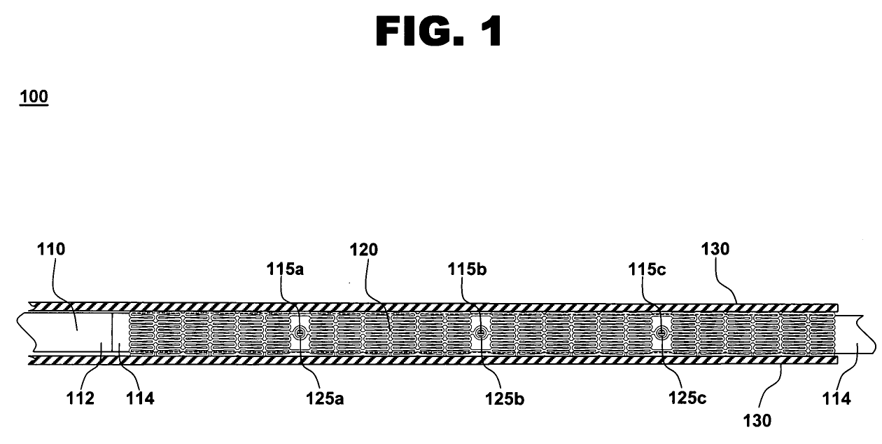

One aspect according to the present invention is a system for treating a vascular condition. One embodiment of the system, in accordance with the present invention, is illustrated at 100 in FIG. 1. The system comprises a catheter inner member 110, a stent 120, and a sheath 130. Inner member 110 has a proximal portion 112 and a distal portion 114, with longitudinally spaced protrusions 115a,b,c, extending from the outer surface of distal portion 114. Stent 120 includes a plurality of longitudinally spaced apertures 125a,b,c, formed in the wall of the stent. Sheath 130 is shown in cross-section to reveal inner member 110 and stent 120 within. Only a distal portion of system 100 is illustrated. As used herein, the terms “distal” and “proximal” are with reference to the treating clinician during deployment of the stent.

Inner member 110 is an elongated structure that, in the present embodiment, includes a central lumen through which a guidewire may pass. Inner member 110 is formed using one or more biocompatible materials such as polyurethane, polyethylene, nylon, or polytetrafluoroethylene (PTFE). The proximal 112 and distal 114 portions of inner member 110 may be formed using the same or different materials. As shown in FIG. 1, the two portions are formed separately and bonded one to the other. Forming the portions separately may provide cost savings and allows the two portions to have different characteristics; for example, it may be desirable for proximal portion 112 to be stiffer than distal portion 114 to ensure pushability of the inner member when delivering stent 120 to a treatment site. In another embodiment, the two portions may be formed from a continuous length of material.

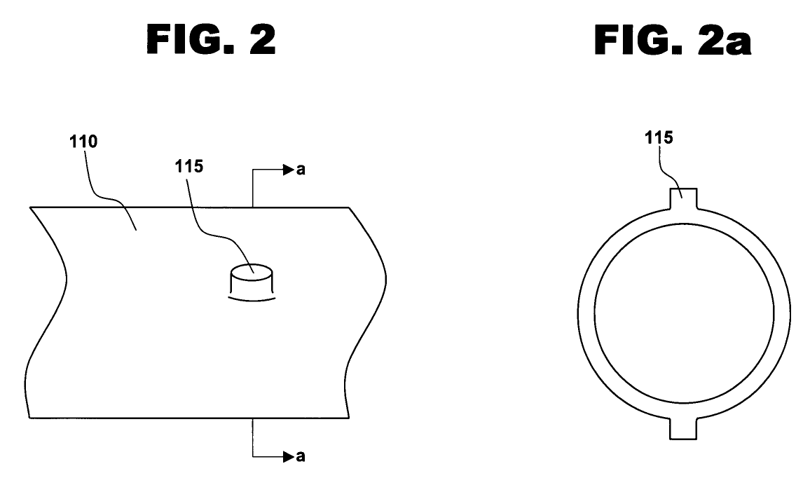

Protrusions 115a,b,c extend from the outer surface of distal portion 114 and are spaced along the length of distal portion 114 (i.e., are longitudinally spaced). Only the top surfaces of protrusions 115a,b,c can be seen in FIG. 1. In the present embodiment, the protrusions are substantially cylindrical as illustrated in FIG. 2, which shows an enlarged view of a single protrusion 115. While FIG. 2A shows a cross section of an end of the inner member taken at a location centered on oppositely configured protrusions. One skilled in the art will appreciate that other shapes are possible, including, but not limited to, elliptical cylinders and polyhedrons. The protrusions may be formed at the same time as the inner member distal portion (e.g., structures molded as an integral part of the inner member) or may be formed separately using the same or a different material and attached to the inner member distal portion (e.g., plastic or metal structures inserted into or bonded onto the surface of the inner member).

Protrusions e.g., 115 are shaped to be received within apertures (e.g., 125) in stent 120 when the stent is mounted on inner member 110 in a radially compressed configuration, as illustrated in FIG. 1. The protrusions are sized such that each inner member protrusion fits fully within its matching stent aperture and does not extend beyond the outer surface of the stent wall when stent 120 is mounted on inner member 110. In the present embodiment, the height of each protrusion above the adjacent inner member cylindrical surface is substantially equal to the thickness of the stent wall. Protrusions e.g., 115 may include radiopaque markers or may be composed of a radiopaque material such as gold, tantalum, or platinum to aid in positioning stent 120 at a treatment site.

Stent 120 is a self-expanding stent formed from, for example, a nickel-titanium alloy, a nickel-cobalt alloy, a cobalt alloy, a thermoset plastic, stainless steel, a stainless steel alloy, a biocompatible shape-memory material, a biocompatible superelastic material, combinations of the above, and the like.

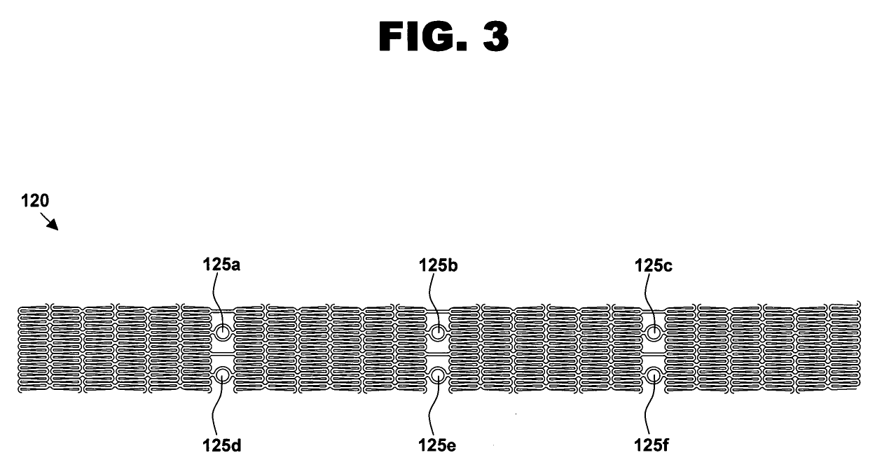

Stent 120 includes a plurality of longitudinally spaced apertures 125a,b,c,d,e,f formed in the wall of the stent. As illustrated in FIG. 3, which shows stent 120 as it would appear if it were cut longitudinally and laid flat, stent 120 has six apertures 125a,b,c,d,e,f. When stent 120 is in its normal cylindrical configuration, the apertures form two sets of three, with one set opposite (i.e., displaced 180 degrees from) the other set. One skilled in the art will appreciate that the number of apertures may vary, with more or fewer apertures being used. In the present embodiment, the number of stent apertures corresponds to the number of inner member protrusions; however, in another embodiment, the number of apertures may exceed the number of protrusions, with only a portion of the apertures receiving protrusions.

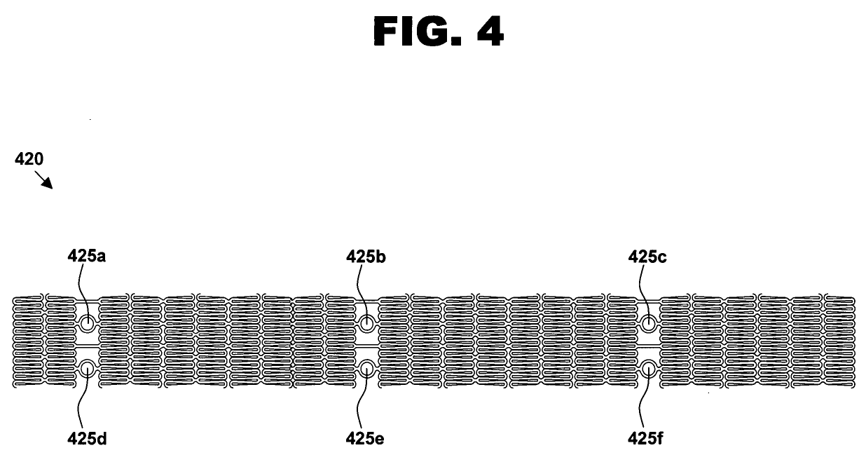

The positioning of the apertures may vary as well. For example, the apertures need not be evenly distributed along the length of the stent as shown in FIG. 3. One alternative spacing is shown in FIG. 4, in which stent 420 includes apertures 425a,b,c,d,e,f that are displaced slightly toward the proximal end of the stent to aid in retaining the stent to the inner member until the stent is fully deployed. A wide variety of other arrangements are possible, including, but not limited to, sets of apertures that are offset from each other on opposite sides of the stent, apertures positioned on one side only of the stent, and apertures distributed around the stent as well as along the length of the stent. The apertures should be positioned to best distribute along the length of the stent forces acting on the stent during deployment, as is discussed more fully below.

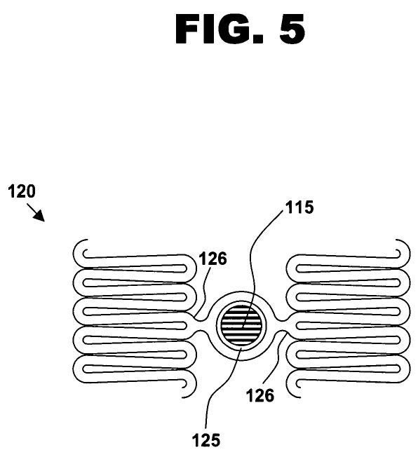

As shown in FIG. 5, apertures e.g., 125 are formed between peak regions (e.g., 126) of segments of stent 120, these peak regions being commonly referred to as “crowns.” FIG. 5 shows an enlarged view of one of the apertures illustrated in FIG. 3, with an inner member protrusion, e.g., 115, received within the aperture e.g., 125. Only the top surface of protrusion 115 is visible.

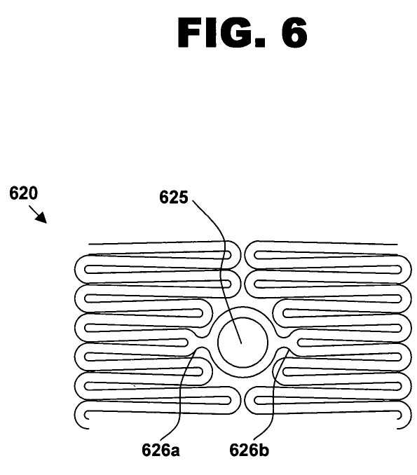

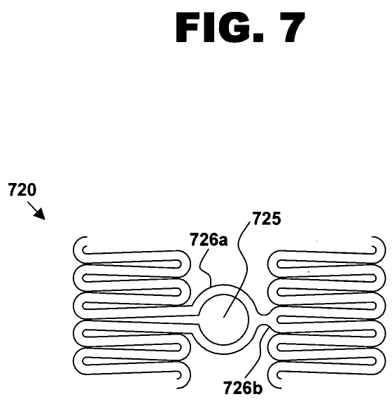

Alternative embodiments of apertures in accordance with the present invention are shown in FIGS. 6 and 7. In FIG. 6, aperture 625 is formed between two shortened crowns 626a and 626b of stent 620, with other crowns of the stent extending to enclose the aperture. In FIG. 7, aperture 725 is formed within one of the crowns, 726a, of stent 725. The crown forming the aperture is extended and enlarged in comparison with the other crowns, e.g. 726b, of the stent.

The stent apertures need not be substantially circular, as shown in FIGS. 1-6, and may assume other shapes depending on the shape of the inner member protrusion to be received within the aperture.

As illustrated in FIG. 1, stent 120 is mounted on inner member distal portion 124 such that inner member protrusions 115a,b,c are received within stent apertures 125a,b,c. Radially compressing stent 120 about inner member 110 effectively interlocks protrusions, e.g., 115, and apertures, e.g., 125.

Sheath 130 having a preset inner and outer diameter encloses stent 120 and a distal portion of inner member 110. Sheath 130 is formed of one or more biocompatible materials. The self expanding stent presses against the inner diameter of the sheath 130. Sheath 130 maintains stent 120 in a compressed configuration and is movable with respect to the inner member 110 so that the sheath may be retracted to allow expansion of the stent 120 that is held by the inner member 110.

Deploying a self-expanding stent involves retracting the enclosing sheath while keeping the stent (and the inner member to which it is attached) stationary at the treatment site. Forces acting on the stent during retraction of the sheath include the radial force of the sheath maintaining the self-expanding stent compressed about the inner member and the axial force resulting from retraction of the sheath. In a stent that is restrained at only the proximal end of the stent throughout the process of withdrawing the sheath, these forces may become concentrated at the proximal end of the stent. This can result in the stent crumpling or buckling as the sheath is withdrawn.

In an embodiment according to the present invention, interlocked inner member protrusions, e.g., 115, and stent apertures, e.g., 125, act as anchoring elements between stent 120 and inner member 110 at multiple intervals along the length of the stent. As sheath 130 is withdrawn, the interlocked protrusions, e.g., 115, and apertures, e.g., 125, act to stabilize the axial motion of each portion of the stent distal to each set of interlocked structures, thereby distributing the deployment force over the length of the stent and preventing longitudinal compression or buckling of stent 120.

As sheath 130 is withdrawn, the portion of stent 120 exposed beyond the end of the sheath 130 expands radially outward from inner member 110, and stent apertures, e.g., 125, move away from inner member protrusions, e.g., 115, releasing stent 120 from inner member 110.

While the system for treating a vascular condition is discussed above in the context of a system that delivers a self-expanding stent, one skilled in the art will recognize that the system may be used for other purposes, for example delivering a self-expanding stent-graft combination. The system may also be useful for delivering a coated stent, the interlocked protrusions and apertures distributing along the length of the stent any additional forces resulting from adhesion of a sheath to the stent coating.

Another aspect according to the present invention is a system for treating a vascular condition comprising a catheter, a stent disposed on the catheter, a sheath releasably enclosing the stent, and means for distributing, along a length of the stent, forces exerted on the stent during release of the stent from the sheath.

In one embodiment in accordance with the present invention, the catheter is a delivery catheter including an inner member such as is described above and illustrated in FIG. 1. The inner member includes protrusions positioned to be received within apertures formed in the wall of the stent. In the present embodiment, the stent is a self-expanding stent as described above and illustrated in FIG. 2. The inner member protrusions and stent apertures collectively serve as means for distributing, along the length of the stent, forces exerted on the stent during release of the stent from the sheath. As discussed above, these forces include a radial force resisting the expansion of the stent from a compressed configuration and an axial force resisting the retraction of the sheath as the frictional force between the stent and the sheath must be overcome to initiate and complete sheath retraction. The protrusion/aperture combinations distribute these forces such that the forces are divided amongst sections of the stent defined by the positioning of the apertures.

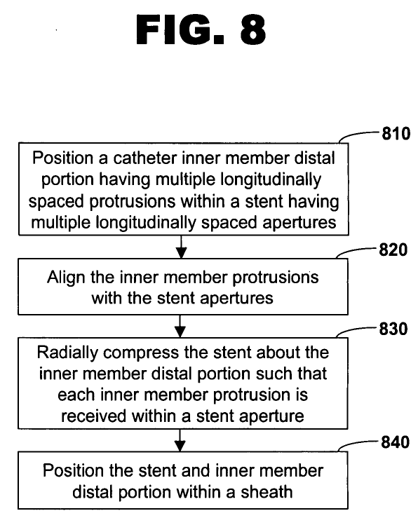

Yet another aspect according to the present invention is a method of assembling a system for treating a vascular condition. FIG. 8 shows a flow diagram of one embodiment of the method in accordance with the present invention.

A catheter inner member distal portion is positioned within a stent (Block 810). The distal portion has a plurality of longitudinally spaced protrusions; i.e., the protrusions are distributed along the length of the distal portion. The stent has a plurality of apertures formed in the wall of the stent and distributed along the length of the stent.

The inner member protrusions are aligned with the stent apertures (Block 820). Alignment may be accomplished by radially compressing the stent to an interim configuration and rotating the inner member until the inner member protrusions engage the stent apertures. The stent may be compressed to the interim configuration either before or after inserting the inner member into the stent. Alternatively, the inner member may be inserted into the fully expanded stent, and the protrusions and apertures may be aligned visually.

The stent is progressively radially compressed about the inner member distal portion such that each inner member protrusion is received within a stent aperture (Block 830). The stent and some or all of the inner member distal portion are enveloped by a sheath (Block 840). The interlocked protrusions and apertures anchor the position of stent relative to the inner member, allowing the stent to be withdrawn from a stent radial compression device (machine) and positioned within the sheath by pulling on a proximal portion of the inner member rather than by pushing on the stent, the inner member, and sheath. Thus, a stent that does not have sufficient column strength or rigidity to be pushed out of the stent compression device may instead be pulled from the device, eliminating the risk of longitudinal compression or buckling of the stent. Alternatively, the stent and inner member portion may be positioned within the sheath using techniques known in the art.

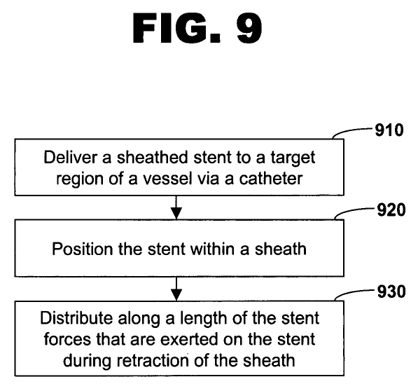

Still another aspect according to the present invention is a method of treating a vascular condition. FIG. 9 shows a flow diagram of one embodiment of the method in accordance with the present invention.

A sheathed stent is delivered to a target region of a vessel via a catheter (Block 910). In the present embodiment, the sheathed stent is a system such as is described above and illustrated in FIG. 1. The stent includes apertures formed in the wall of the stent that are spaced along the length of the stent. The apertures receive, and are effectively interlocked with, protrusions extending from a distal portion of an inner member about which the stent is compressed.

The sheath is retracted from the stent (Block 920). Forces exerted on the stent during retraction of the sheath are distributed along the length of the stent (Block 930). These forces include the radial force of the sheath maintaining the self-expanding stent compressed about the inner member and an axial force resulting from retraction of the sheath. The interlocked stent apertures and inner member protrusions anchor the stent to the inner member at multiple intervals along the length of the stent. As the sheath is withdrawn, the interlocked apertures and protrusions act as anchors to resist the effect of the deployment forces to a portion of the stent distal to a set of interlocked structures, thereby distributing the deployment forces over the length of the stent and preventing longitudinal compression or buckling of the stent. When the stent apertures are evenly distributed along the length of the stent, as in the present embodiment, the forces associated with deployment are distributed equally along the length of the stent.

While the embodiments of the invention are disclosed herein, various changes and modifications can be made without departing from the spirit and scope of the invention.

What is claimed is:

1. A system for treating a vascular condition, comprising:
a catheter inner member having a proximal portion and a distal portion, the distal portion having a plurality of longitudinally spaced protrusions extending from an outer surface of the distal portion;
a stent having a plurality of longitudinally spaced apertures formed in a wall of the stent, the stent mounted on the inner member such that the inner member protrusions are received within the stent apertures; and
a sheath movably enclosing the stent.

2. The system of claim 1 wherein the inner member protrusions are formed as an integral part of the inner member distal portion.

3. The system of claim 1 wherein the inner member protrusions are formed separately and attached to the inner member distal portion.

4. The system of claim 1 wherein at least a portion of each inner member protrusion is radiopaque.

5. The system of claim 1 wherein the inner member distal portion is bonded to the inner member proximal portion.

6. The system of claim 1 wherein the apertures are formed between crowns of the stent.

7. The system of claim 1 wherein the apertures are formed within crowns of the stent.

8. The system of claim 1 wherein the stent apertures are evenly distributed along the length of the stent.

9. The system of claim 1 wherein the height of each inner member protrusion over the adjacent inner member surface is substantially equal to the thickness of the stent wall.

10. The system of claim 1 wherein the number of stent apertures corresponds to the number of inner member protrusions.

11. The system of claim 1 wherein the stent is a self-expanding stent.

12. The system of claim 1 wherein the stent comprises a material selected from a group consisting of a nickel-titanium alloy, a nickel-cobalt alloy, a cobalt alloy, a thermoset plastic, stainless steel, a stainless steel alloy, a biocompatible shape-memory material, a biocompatible superelastic material, and a combination thereof.

13. A method of assembling a system for treating a vascular condition, the method comprising:
positioning a catheter inner member distal portion having a plurality of longitudinally spaced protrusions within a stent having a plurality of longitudinally spaced apertures;
aligning the inner member protrusions with the stent apertures;
radially compressing the stent about the inner member distal portion such that each inner member protrusion is received within a stent aperture; and
positioning the stent and at least a portion of the inner member distal portion within a sheath.

14. The method of claim 13 wherein aligning the inner member protrusions with the stent apertures comprises:
radially compressing the stent to an interim configuration; and
rotating the inner member until the inner member protrusions engage the stent apertures.

15. The method of claim 14 wherein the stent is radially compressed to an interim configuration prior to positioning the catheter inner member within the stent.

16. The method of claim 13 wherein positioning the stent within a sheath comprises pulling on a proximal portion of the inner member to position the stent within the sheath.

17. A method of treating a vascular condition, the method comprising:
delivering a sheathed stent to a target region of a vessel via a catheter;
retracting a sheath from the stent; and
distributing along a length of the stent forces exerted on the stent during retraction of the sheath;
wherein the forces are distributed as a result of the interlocking of longitudinally spaced apertures formed in a wall of the stent and protrusions extending from a catheter inner member distal portion received within the stent.

18. The method of claim 17 wherein the forces are distributed equally along the length of the stent.
